(19) United States
(12) Patent Application Publication
(10) Pub. No.: US 2002/0091348 A1
(43) Pub. Date: Jul. 11, 2002

(54) Training system for joint disorders and other uses

(76) Inventor: Mark Joseph, Aspen, CO (US)

Correspondence Address: GLENN L WEBB, P.O. BOX 951, CONIFER, CO, 80433

(21) Appl. No.: 09/975,597

(22) Filed: Oct. 10, 2001

Related U.S. Application Data
(60) Provisional application No. 60/250,003, filed on Nov. 30, 2000.

(51) Int. Cl. A61L 15/00

(52) U.S. Cl. 602/75

(57) ABSTRACT

A system and process for training the user regarding undesired rotation of body joints. The system and process uses an elongated flexion strip that includes an adhesive layer. The flexion strip is attached on the skin of the user surrounding the body joint. The flexion strip is situated so that if the user attempts to rotate the body joint to an undesired position, the skin is pulled to cause mild discomfort to the user. This provides negative reinforcement to the user to train the user not to move that body joint in an undesired range of motion. This system and process can be used not only to treat disorders or injuries but for training for performance activities as well.

### RELATED APPLICATIONS

This application relates to the subject matter of provisional application 60/250003, filed on Nov. 30, 2000.

### FIELD OF THE INVENTION

This invention relates to the field of systems and processes for treating joint disorders, performance activities and other uses.

### BACKGROUND OF THE INVENTION

A number of disorders are generally associated with repetitive and forceful use of the hands that damage muscles and bones of the upper extremities and can cause compression neuropathy. These disorders are categorized by several different terms, such as repetitive stress injuries, cumulative trauma disorder, overuse syndromes, chronic upper limb pain syndrome or repetitive motion disorders. One type of injury that results from these disorders are related nerve entrapment syndromes such as carpal tunnel syndrome and ulnar nerve syndrome.

Carpal tunnel syndrome and ulnar nerve syndrome are very similar in nature. The carpal tunnel is a passageway that runs from the forearm through the wrist. Bones form three walls of this tunnel that is bridged by a strong, broad ligament. The median nerve that supplies feeling to the thumb, index, and ring fingers, and the nine tendons that flex the fingers pass through this tunnel. Repetitive stress, injury, or other conditions may cause the tissues around the median nerve to become swollen, either by the ligament or by the protective sheath surrounding the tendons. This causes compression on the median nerve fibers and results in slowing of the transmission of nerve signals through the carpal tunnel. The result is pain, numbness and tingling in the wrist, hand and related fingers. The ulnar nerve travels through a similar tunnel, called the ulnar tunnel that lies between the bones of the elbow surrounded by a ligament. As this ligament hardens, the nerve is pressed into the small space of this tunnel. This results in pain, numbness and tingling in the little finger and ring finger of the hand.

Often, these types of disorders or injuries are exacerbated during sleep. Many individuals naturally curl into a fetal-like position during their sleep patterns that may cause the joint in question to be further inflamed or injury. Presently, the only non-surgical technique to prevent this is to use braces or splints. This often is uncomfortable and disrupts the sleep of the individual.

The current treatment for these disorders includes surgical and non-surgical options. The non-surgical options are presently limited in scope and effectiveness. One non-surgical option is to utilize braces or splints over the affected joint to prevent the joint from bending. This is intended to allow the swollen and inflamed synovial membranes to shrink and thus relieve pressure on the nerve. However, this particular treatment may actually exacerbate the condition. Splints or braces worn during the day decrease the muscle pumping action of the hand. This in turn may cause more swelling in the tunnel and increase the disorder. Splints or braces may be helpful at night, but tend to be awkward and affect sleeping patterns.

Examples of the above type attempts at solving these problems are disclosed in U.S. Pat. Nos. 6,213,969; 5,417,645; 6,063,087; 5,328,446; 5819,313; 6,106,492; 6,120,472; 5,865,783; 5,766,141; 5,919,151; 5,921,949; 5,746,707; 6,146,347; 6129,692; 5,925,007 and many others. None of these actually retrain the user from undesired joint movement.

Another similar problem exists in performance activities. Many activities that utilize specific performance of the joints, such as musical instruments, sports activities, surgical procedures and others, require particular and specific movements in order to perform successfully. Also, incorrect movement of these joints may lead to injuries as well as the disorders discussed above. Presently, these movements must be specifically taught and monitored to discourage incorrect movement.

There presently exists a need for a system to minimize the movements that result in compression neuropathy syndromes, that alleviate the tissue swelling that cause such injuries and that “retrain” the movement of the joints that cause such syndromes. Also, there presently exists a need for a training system for training the muscles and joints of an individual for specific movements.

### SUMMARY OF THE INVENTION

The present invention provides systems and processes for minimizing further injury to disorders and injuries to or resulting from movement of body joints, such as wrists, elbows, fingers, knees, ankles, hips and others and for training the user from repeating movements of those body joints in undesired range of motions.

The preferred embodiments of the present invention may also be used to train users from undesired motions for not only work-related activities, or lifestyle activities but also for performance activities such as music or sports.

The preferred embodiment of the present invention provides an elongated flexion strip of inelastic material. Other embodiments may utilize elastic material as well. The shape and size of the flexion strip may be chosen for body specific applications. In one preferred embodiment, intended for training the user from specific ranges of motions of wrist movement to alleviate carpal tunnel syndrome, the flexion strip includes two finger extensions. The flexion strip is of sufficient length to extend partially along the forearm of the user over the wrist joint. In this preferred embodiment, the flexion strip includes an adhesive layer for attaching the flexion strip onto the skin of the user.

In use, the finger extensions are applied to the ring finger and middle finger of the user and extends over the wrist joint and up the forearm. The adhesive layer attaches the flexion strip directly onto the skin of the user. The flexion strip is applied so that the hand and forearm are in substantially the same plane. If necessary, the hand may be bent slightly at the time of application to allow for certain activities.

Once the flexion strip is in place, negative reinforcement is supplied to the user when undesired movement occurs. This negative reinforcement is provided by the pulling of the skin where the flexion strip is attached. This is normally sufficient to discourage this movement. Over time, the user is trained not to allow this movement to occur. The user may go forward with this motion if critically necessary by suffering the mild discomfort.

The user may use a barrier film in another preferred embodiment between the adhesive layer and the skin. This reduces the pain and aggravation of removing the flexion strip while still allowing adequate adhesion of the flexion strip and skin.

This system minimizes and retrains the user from repetitive joint motions that may aggravate disorders resulting from the movement. Also, the system can be used to train or retrain users in performance activities such as musical instruments or sports activities.

The system can be applied not only for wrist motions but for finger motions, elbow motions, knee motions, ankle motions and almost any other body joint.

These and other features of the present invention will be evident from the detailed description of preferred embodiments and from the drawings.

### BRIEF DESCRIPTION OF THE DRAWINGS

FIG. 1 is a perspective view of the flexion strip of a preferred embodiment of the present invention.

FIG. 2 is a perspective view of the embodiment of FIG. 1 applied to a user.

FIG. 3 is a perspective view of the embodiment of FIG. 1 showing discomfort applied to the user.

FIG. 4 is a cross-sectional view of FIG. 2.

FIG. 5 is a side view of the embodiment of FIG. 1 applied to a finger in a normal position.

FIG. 6 is a view of the embodiment of FIG. 5 showing the finger in a position of movement.

FIG. 7 is a view of another embodiment of the present invention.

FIG. 8 is a view of another embodiment of the present invention.

FIG. 9 is a view of another embodiment of the present invention.

FIG. 10 is a view of another embodiment of the present invention.

FIG. 11 is a view of another embodiment of the present invention.

FIG. 12 is a view of another embodiment of the present invention.

FIG. 13 is a view of another embodiment of the present invention.

FIG. 14 is a view of an alternative application of the present invention.

FIG. 15 is a view of an alternative application of the present invention.

### DETAILED DESCRIPTION OF THE SPECIFIC EMBODIMENTS

The present invention provides a training system for treating joint disorders and other uses. It is to be expressly understood that the descriptive embodiments set forth herein are intended for explanatory purposes and is not intended to unduly limit the scope of the claimed inventions. Other embodiments and applications not described herein are considered to be within the scope of the invention. It is also to be expressly understood that while specific embodiments for the components of the training systems are discussed, other equivalents to these embodiments that perform substantially similar functions are within the scope of the claimed inventions.

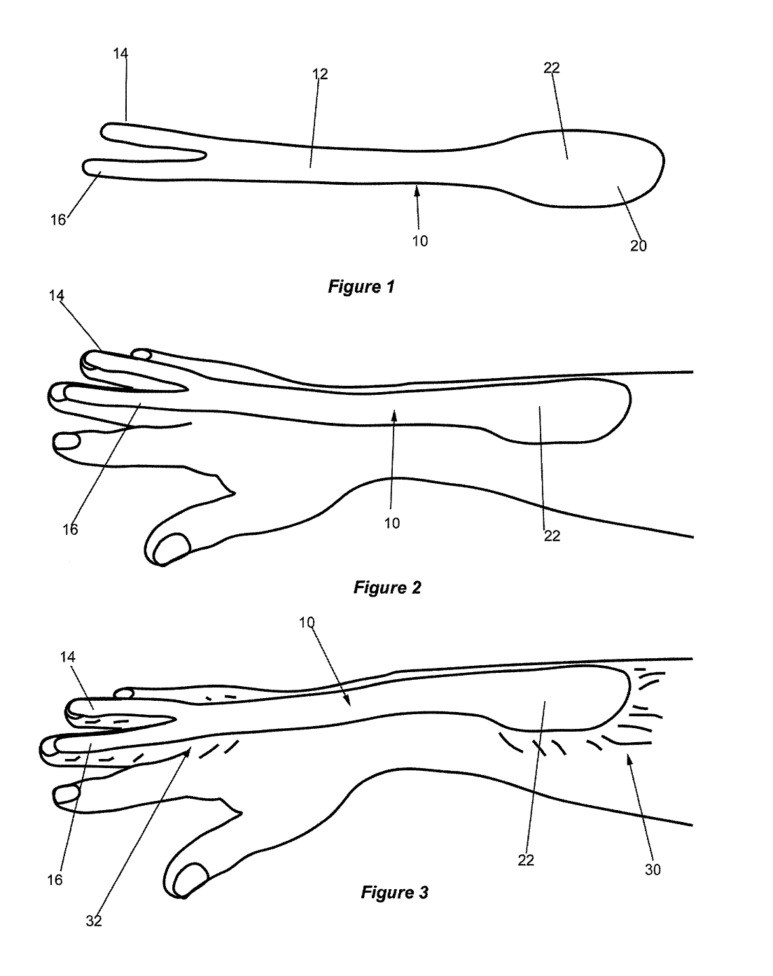

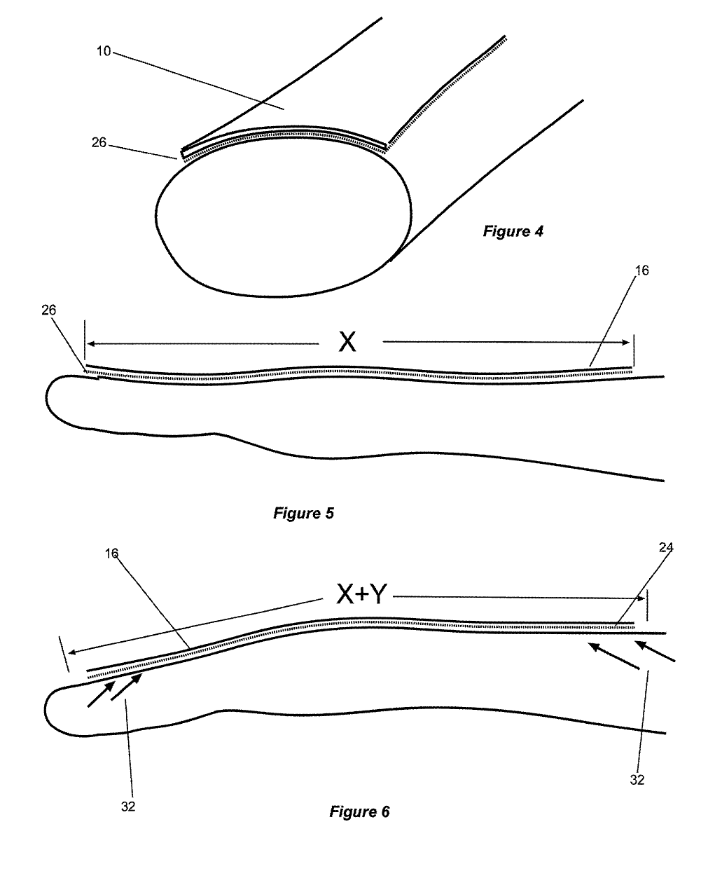

A preferred embodiment of the present invention is illustrated in FIGS. 1-3. The training system of this preferred embodiment does not support or positively constrain movement of the joint being treated in the manner of a brace or splint. Instead, the training system of this preferred embodiment provides negative reinforcement to “train” the joint from incurring the inappropriate movement. In this manner the muscles and associated “memory” relating to this joint are trained to develop appropriate motion and posture.

In this preferred embodiment, the training system includes a flexion limiting flexion strip 10. In this preferred embodiment, flexion strip 10 includes a breathable inelastic backing layer 12, such as found in surgical tape. It is to be expressly understood that in other preferred embodiments under the scope of the present invention that elastic layers could be used as well as non-breathable layers. In this embodiment, however, the combination of a layer that is breathable is desired for comfort and inelastic for effectiveness is preferred.

The flexion strip 10, in this preferred embodiment, is intended to use for wrist related disorders, such as carpal tunnel syndrome. Two finger extensions 14, 16 extend outwardly from a distal end 18 of the flexion strip 10 as shown in FIGS. 1-3. On the opposing proximal end 20, a wider end portion 22 is formed. The elongated center portion 24 extends a distance proportional to the forearm of the typical individual for which it is intended. It is to be expressly understood that other shapes can be used, particularly for use with other body joints.

An adhesive layer 26 lies underneath the backing layer 12. This adhesive layer is relatively aggressive to bond onto the outer skin of the user but sufficiently mild so not damage the skin. A release backing 28 is applied over the adhesive layer until the flexion strip is utilized. In the preferred embodiment, the release backing is die cut at the distal end to improve ease of application.

The flexion strip 10 is applied, in this preferred embodiment, to apply pressure and mild discomfort when the wrist of a user is moved to an undesirable position. This pressure and mild discomfort may in many applications be desired to train the wrist from use that resulted in a repetitive stress injury, such as carpal tunnel syndrome. Other applications may be to train the user from moving the wrist to an incorrect position, such as in musical performances, sports trainings or other uses.

The flexion strip is applied as follows: the release backing 28 is initially removed from the distal end 18 of the flexion strip 10. The finger extensions 14 and 16 are applied against the ring and middle fingers (or other fingers) of the user so that the adhesive bonds against the skin. The remainder of the release backing is removed from the flexion strip so that that the flexion strip can be adhered against the forearm of the user. The larger end portion 24 of the flexion strip provides greater adhesion areas to enhance the discomfort. The placement of the flexion strip is important in the training process. For example, most users will place the flexion strip so that it adheres to the forearm and back of the hand as the hand and forearm are straight or slightly flexed. Of course, if further movement of the hand relative to the forearm at the wrist is desired, this can be accommodated at the time of placement of the flexion strip.

In use, the user has the ability to use their wrist in normal or desired positions. If the user attempts to rotate their wrist into an undesired position, the flexion strip will cause a mild discomfort from the inelasticity of the flexion strip and adhesive bonding to the skin. This is shown by arrows 30, 32 in FIGS. 3 and 6. If necessary, the user can overcome this discomfort to rotate their wrist further. However, the mild discomfort provides negative reinforcement to minimize incidental or accidental movement of the wrist in undesired positions. This negative reinforcement over time will train the user not to make such movements.

This particular preferred embodiment can also be used not only for wrist movements but for finger movements as well. The finger extensions 14, 16 may be secured over the knuckles of the fingers to minimize their movement as well. The same process occurs. Mild discomfort is created from the inelasticity of the flexion strip and the adhesive bonding with the skin if the fingers are moved beyond their normal state as shown in FIGS. 5 and 6. The initial position of the flexion strip 10 has a length “X”. Upon rotation of the finger knuckle, the flexion strip attempts to elongate to accommodate this movement to a length “X+Y”. This causes the adhesive to pull the skin at arrows 32 that creates discomfort to the wearer.

This process of “training” the joint being treated has applications beyond that of treating disorders or injuries. It may also be used in training the joint and the user in performance activities. For example, the correct placement and movement of the wrist and fingers are critical in musical instruments such as violin. The incorrect placement and movement of the wrist and fingers not only may impact the performance of the instrument but also lead to later injuries. The present invention may also be used for other joints, such as elbows, knees, hips, ankles and other joints. Also, this process may be used in training athletes for different sports. An example may be training a baseball pitcher from

In another preferred embodiment, a barrier film may be used to minimize the damage to the skin from the adhesive bonding. One such barrier film is produced by 3M Company, under the trade name Cavilon™. This barrier film still allows the flexion strip to be securely bonded to the skin while lessening the pain and damage from removal of the flexion strip from the skin.

The present invention also includes many other embodiments that may be used depending on the joint, the disorder, the activity or other factor. For example, a flexion strip having a longer mid section and without the finger extensions may be used to minimize movement of the elbow joint for ulnar tunnel syndrome or for other injuries concerning the elbow. Other shapes may be used to treat disorders or activities concerning the knee, the ankle, the hip or other joints. Differing shapes may also be used to treat differing sizes of individuals, ranging from infants to adults. The different embodiments may also use tabs, wraps, and other techniques for fastening the flexion strip to the individual.

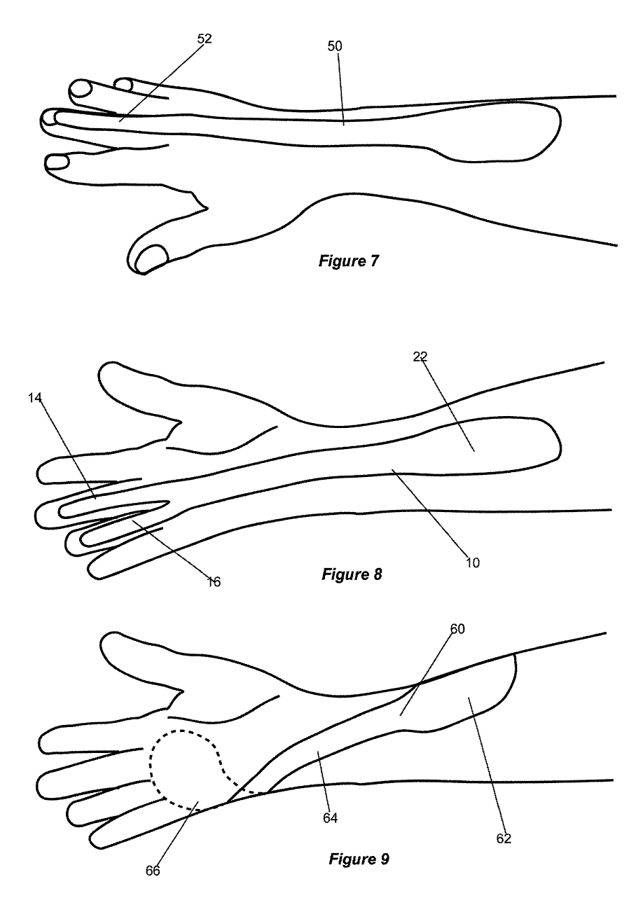

Examples of various embodiments are illustrated in FIGS. 7-15. FIG. 7 illustrates an embodiment that uses only a single finger extension. Another embodiment, shown in FIG. 8, uses a flexion strip similar to the above described flexion strip but applied on the inner arm, inner wrist and inner hand and fingers. This is used to discourage backward rotation of the wrist. Another embodiment as shown in FIG. 9 uses larger portions at the ends of the flexion strip and is applied, for example, to discourage side to side rotation of the wrist or other joints.

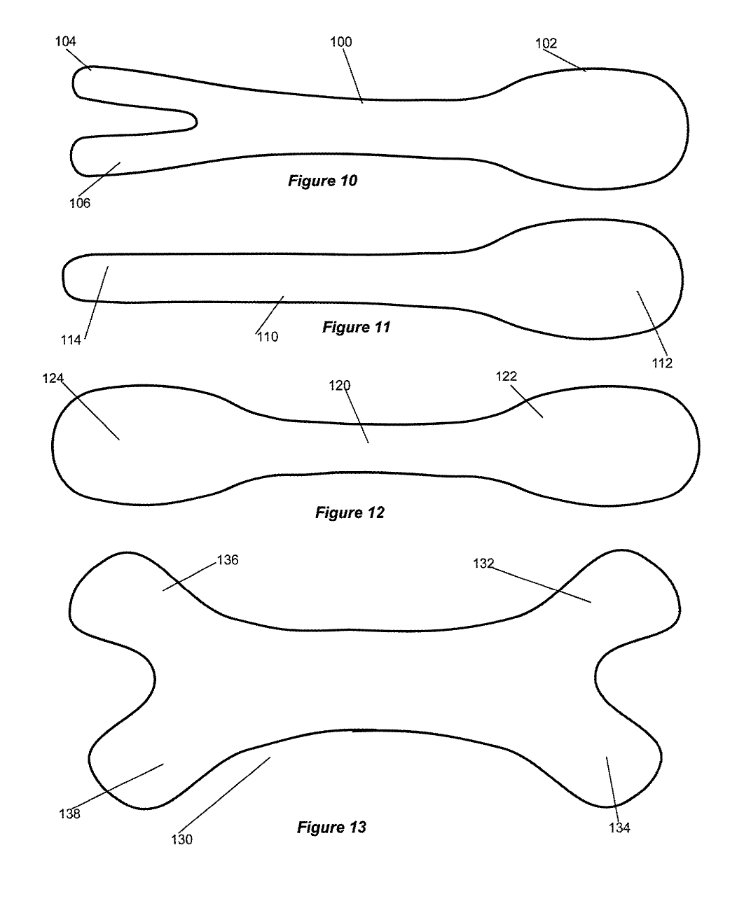

FIG. 10 shows another embodiment 100 having a large end portion 102 and two extension members 104, 106. Yet another embodiment 110 shown in FIG. 11 uses a large end portion 112 and a single smaller end portion 114. FIG. 12 shows a flexion strip 120 having opposing large end portions 122, 124. FIG. 13 shows a flexion strip 130 having two extension members 132, 134 on one end and two extension members 136, 138 on the opposing end. Other shapes and configurations may be used as well for different applications. Often, larger areas, such at the ends, are desired to increase the areas of discomfort.

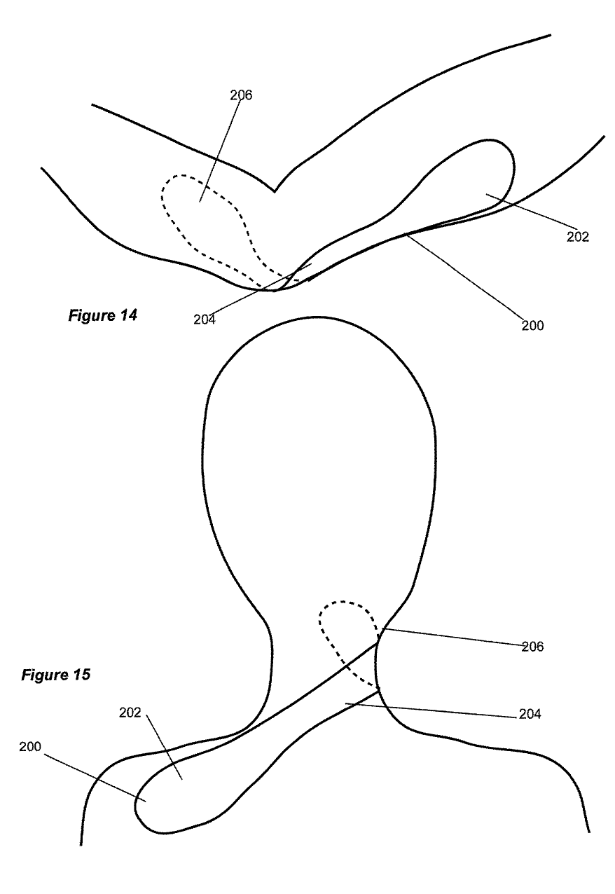

These flexion strips may be used for various applications other than the wrist applications discussed above. For example, as shown in FIG. 14, flexion strip 200 may be used to discourage inappropriate elbow rotation by applying end 202 on the forearm and end 206 at the tricep area. Mid portion 204 is applied over or near the elbow. Another application to discourage neck rotation is shown in FIG. 15. End 202 of flexion flexion strip 200 is applied on the upper back or neck region while end 206 is applied on fore portion of the neck. These applications discourage inappropriate movement by causing discomfort.

These and other embodiments of this system and processes are considered to be within the scope of the claimed inventions. The above descriptive embodiments are provided for explanatory purposes only and are not meant to limit the scope of the claimed inventions.

What is claimed is:

1. A system for training the movement of joints, said system comprising:
a substantially elongated flexion strip;
a first mechanism for attaching a first portion of said flexion strip to a first portion of an individual near a body joint;
a second mechanism for attaching a second portion of said flexion strip to a second portion of an individual near said body joint; and
said flexion strip minimizing the ability of said body joint to rotate.

2. The system of claim 1 wherein said substantially elongated flexion strip includes:
a substantially inelastic portion.

3. The system of claim 1 wherein said substantially elongated flexion strip includes:
a substantially elastic portion.

4. The system of claim 1 wherein said first mechanism includes:
an adhesive layer for adhering onto the skin of a user.

5. The system of claim 1 wherein said second mechanism includes:
an adhesive layer for adhering onto the skin of a user.

6. The system of claim 1 wherein said system further includes:
an adhesive layer for securing said flexion strip to the skin of a user; and
a barrier film for protecting the skin while allowing said flexion strip to adhere to the skin of a user.

7. A system for training the movement of a body joint, said system comprising:
a substantially elongated flexion strip;
an adhesive layer for attaching said flexion strip to the skin of a user adjacent a body joint; and
a release backing for protecting said adhesive layer from accidental adhesion.

8. A process for training the movement of a body joint, said process comprising the steps of:
providing a substantially elongated flexion strip;
attaching said substantially elongated flexion strip near one end of said body joint on the surface of the skin of a user; and
attaching said substantially elongated flexion strip near one end of said body portion near the opposing end of said body joint on the surface of the skin of the user to provide discomfort to said user when said body joint rotates into an undesired position.

9. The process of claim 8 wherein said steps of attaching said substantially elongated flexion strip includes:
providing an adhesive layer on said substantially elongated flexion strip.

10. The process of claim 9 wherein said process further includes: providing a barrier film to minimize damage to the skin of the user while allowing adhesion of said flexion strip to the skin.
